(19) United States
(12) Patent Application Publication
(10) Pub. No.: US 2001/0007929 A1
(43) Pub. Date: Jul. 12, 2001

(54) Human joint bandage

(75) Inventor: Jens Schlomski, Duderstadt, DE (US)

Correspondence Address: Robert L. Muir, Husch & Eppenberger, LLC, 401 Main Street, Suite 1400, Peoria, IL, 61602-1741, US

(73) Assignee: Otto Bock Orthopadische Industrie GmbH & Co.

(21) Appl. No.: 09/755,344

(22) Filed: Jan. 5, 2001

(30) Foreign Application Priority Data
Jan. 7, 2000 (DE) 200 00 265.1
Mar. 16, 2000 (DE) 100 12 984.6

(51) Int. Cl. A61F 13/00; A61F 13/06

(52) U.S. Cl. 602/63; 602/62

(57) ABSTRACT

An elastic hose-shaped bandage for pulling over a human joint having a front side and a bending side wherein the front side is approximate to the joint and bending side is approximate to the hollow of the joint. The bending side has a twin-layer knitted spacer fabric for conducting moisture away from the patient's skin. The spacer fabric comprises two spaced layers and connecting threads.

### FIELD OF THE INVENTION

This invention relates generally to the field of medical bandages and more specifically to a bandage which provides protection, support or splinting for a strained, dislocated, deformed, unstable, arthritic or fractured joint.

### DESCRIPTION OF THE RELATED ART

Bandages are used for medical purposes and for supporting human joints, e.g. knees, elbows, etc. The bandage is pulled over the human joint. For example, the bandage can be a knee joint bandage or ortheses used for supporting unstable knee joints during the conservative post-operative treatment of ligament ruptures, reducing the pain caused by arthritic knees, or supporting the knee after lateral ligament injuries. The knee joint bandage is pulled over the knee joint of the patient and worn daily during treatment and especially during walking and running. A drawback with conventional knee joint bandages is that they produce folds in the region of the hollow of the knee during walking or running which can strangulate the knee bend in the bending position. Conventionally manufactured knee joint bandages are almost entirely made by flat or round knitting which leads to sweating in the bend of the knee of the patient. The sweating can cause a raw wound to develop on the knee.

The present invention is directed to overcoming one or more of the problems set forth above.

### SUMMARY OF THE INVENTION

An object of the invention is to provide an improved joint bandage for pulling over a human joint.

It is another object of the invention to provide a bandage which is economical to manufacture.

In one aspect of the invention there is provided an improved bandage that has a twin-layer knitted spacer fabric on the bending side of the bandage for conducting moisture.

In another aspect there is provided a bandage having a twin-layer moisture-conducting knitted spacer fabric for transporting away from the skin sweat that accumulates in the bending region of a human joint.

In still another aspect there is provided a bandage having a knitted structure which precludes formation of large folds in the joint bandage when the joint is in bent position.

The manufacture of the joint bandage is simplified when the knitted spacer fabric extends over the full length of a hose. In this case the hose is formed of tow knitted pieces of different width which are joined into the hose. The joining is preferably effected by two flat seams.

In another aspect there is provided a bandage having a knitted spacer fabric that is widened in a central section relative to the longitudinal direction to improve the moisture transport especially in the region of the joint bend of the patient or user.

It is particularly advantageous when the knitted spacer fabric is provided in an opening which, in relation to the longitudinal direction, is provided in a central section. The opening on the bending side is arranged so that, after being pulled on, the joint bandage is positioned in the joint bend. Preferably the opening goes at least halfway around the hose. The construction of the opening has the effect that the area producing folds in the bent position of the joint, transverse to the longitudinal direction of the bandage, is completely covered by the moisture-conducting knitted fabric inserted in the opening. Preferably the two layers of knitted spacer fabric run essentially parallel to each other and are spaced by moisture-conducting threads.

In another aspect there is provided a bandage having a knitted spacer fabric in the area of the joint bend for transporting moisture from the layer directly in contact with the skin of the patient via the moisture-conducting connecting threads to the layer of the knitted fabric remote from the body running substantially parallel to the layer in contact with the skin.

The above are merely illustrative and should not be construed as all-inclusive.

### BRIEF DESCRIPTION OF THE DRAWINGS

Reference is now made more particularly to the drawings which illustrate the best presently known mode of carrying out the invention and wherein similar reference characters indicate the same parts throughout the views.

FIG. 1 is a front view of an elastic hose-shaped joint bandage;

FIG. 2 is a rear view illustrating the bending side of the elastic hose-shaped joint bandage;

FIG. 2a is a rear view illustrating a second embodiment of the bending side of the elastic hose-shaped joint bandage having a knitted spacer fabric with an expanded width;

FIG. 2b is a rear view illustrating a third embodiment of the bending side of the elastic hose-shaped joint bandage;

FIG. 3 is a side view of the elastic hose-shaped joint bandage embodiment according to FIG. 2;

FIG. 3a is a side view of the elastic hose-shaped joint bandage embodiment according to FIG. 2b; and

FIG. 4 is an enlarged sectional view of the knitted spacer fabric taken at IV in FIG. 3.

### DETAILED DESCRIPTION

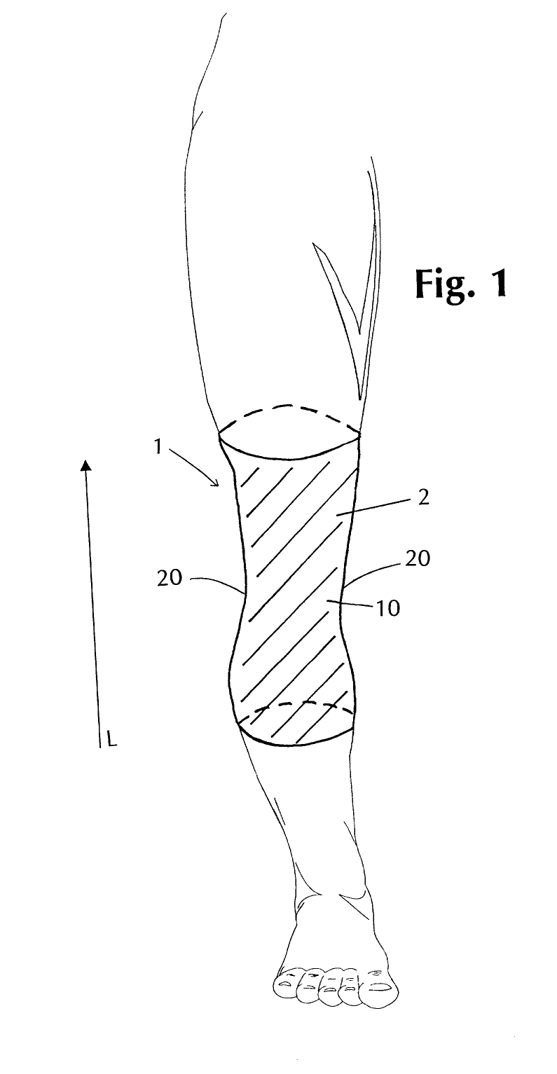

FIG. 1 illustrates an elastic hose-shaped joint bandage, generally designated 1, having a flat knitted fabric 10 piece knitted in a right-right linkage. The flat knitted fabric 10 includes approximately one half synthetic fibers such as crimped PERLON® and/or NYLON®, and the other half of rubber and elastic fibers. PERLON® is a federally registered trademark of Perlon-Warenzeichenverband E.V. Association. NYLON® is a federally registered trademark of Nylon, L.L.C. In a preferred embodiment the yarns used are 50% crimped synthetic fibers (Helanca PH); 46% rubber threads; and 4% elastic fibers. Helanca is a NYLON® like material which can be pulled in any direction and still return to its original shape. The materials that are used to manufacture the flat knitted fabric 10 are selected to achieve an advantageous relationship between elasticity for adapting to the changing joint form as a result of movement, and stability for supporting the joint.

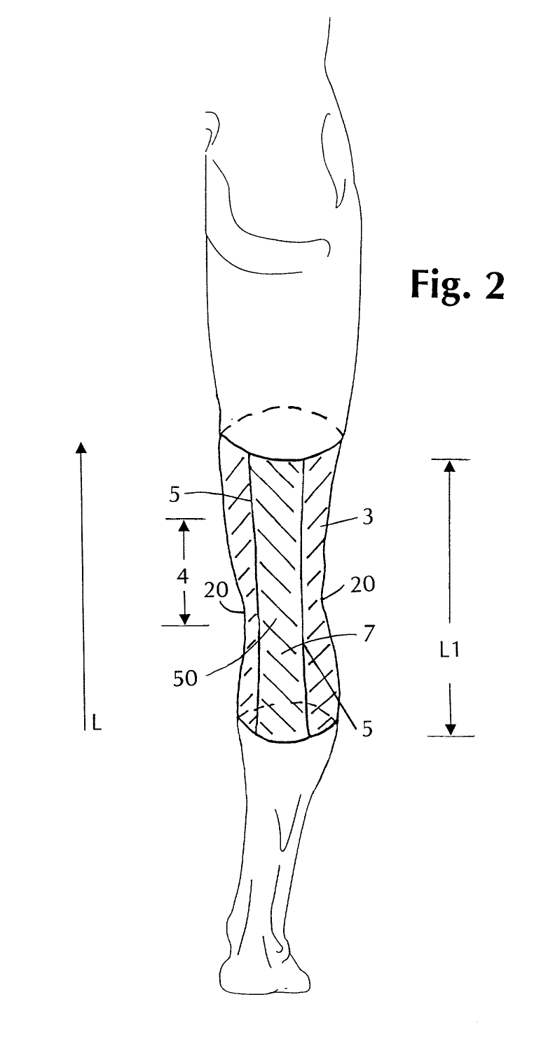

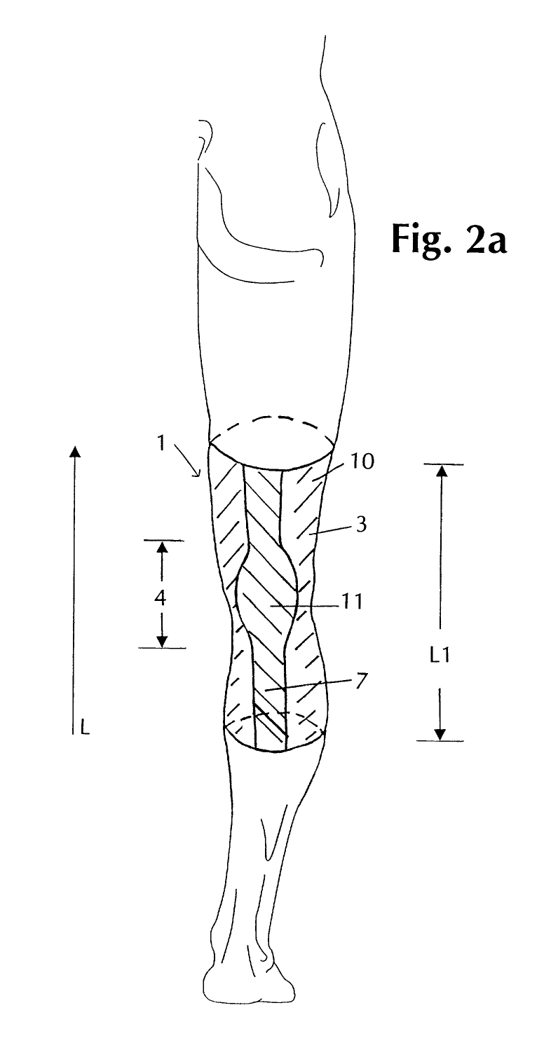

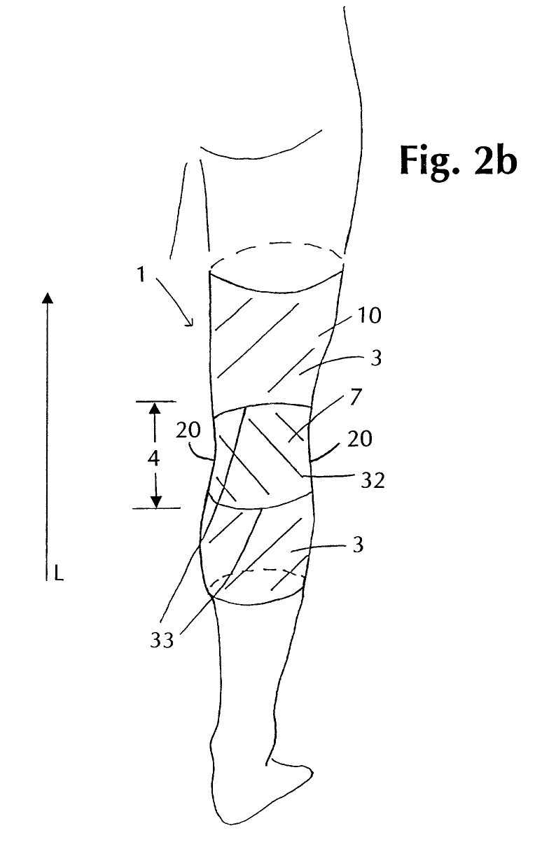

Referring now to FIG. 2, the bandage 1 has two sections, the flat knitted fabric 10 and a strip-like twin-layer knitted spacer fabric 7. The strip-like twin-layer knitted spacer fabric 7 conducts moisture away from the patient's skin. The strip-like twin-layer knitted spacer fabric 7 and flat knitted fabric 10 have different widths and are joined together at flat seams 5. The bandage 1 is similar to an elastic hose having a front side 2 and a bending side 3 which are arranged opposite in their projected area. The bandage 1 can be pulled in a longitudinal direction L over various joints of the human body such as a knee or elbow joint. In the case of a human leg, the front side 2 extends over the knee cap, and the bending side 3 extends over the hollow 50 of the knee. The bending side 3 has the strip-like twin-layer knitted spacer fabric 7 for conducting moisture. The materials used to manufacturer the strip-like twin-layer knitted spacer fabric 7 is discussed hereinafter. The remainder of the bandage 1 is advantageously made of the flat knitted fabric 10. The bandage 1 is substantially rectangular with a central section 4 and has longitudinal sides 20 which are curved toward each other. The bandage 1 is advantageously constructed so that it is in tight contact with the limb over the maximum effective area.

The bandage 1 has a length L1 and the strip-like twin-layer knitted spacer fabric 7 extends over the entire length L1 on the bending side 3. The strip-like twin-layer knitted spacer fabric 7 is attached to the flat knitted piece 10 via the flat seams 5. This strip-like twin-layer knitted spacer fabric 7 conducts moisture from the area between the bandage 1 and the skin of the patient. The strip-like twin-layer knitted spacer fabric 7 absorbs the sweat collecting in the bending region and transports the moisture away from the skin. In this FIG. 2 embodiment there is no flat knitted piece 10 in the hollow 50 of the knee, and the strip-like twin-layer knitted spacer fabric 7 precludes formation of large folds in the joint bandage when bending at the joint.

FIG. 2a illustrates a second embodiment of the bandage 1 having the strip-like twin-layer knitted spacer fabric 7. However, the strip-like twin-layer knitted spacer fabric 7 is shown having an expanded width 11 in the central section 4. The expanded width 11 is designed to cover the maximum area of the bending joint, or in the case of a knee, the hollow of the knee 50 on the bending side 3. The expanded width 11 in the central section 4 improves the conducting of moisture away from the skin of the patient. The expanded width 11 of the strip-like twin-layer knitted spacer fabric 7 covers the area of the bending side 3 of the knee most prone to moisture or sweating so that the moisture or sweat can be transported away from the skin of the patient to prevent discomfort, rash, rawness, wounds or odor. The structure of the strip-like twin-layer knitted spacer fabric 7 will be described in further detail hereinafter and with respect to FIG. 4.

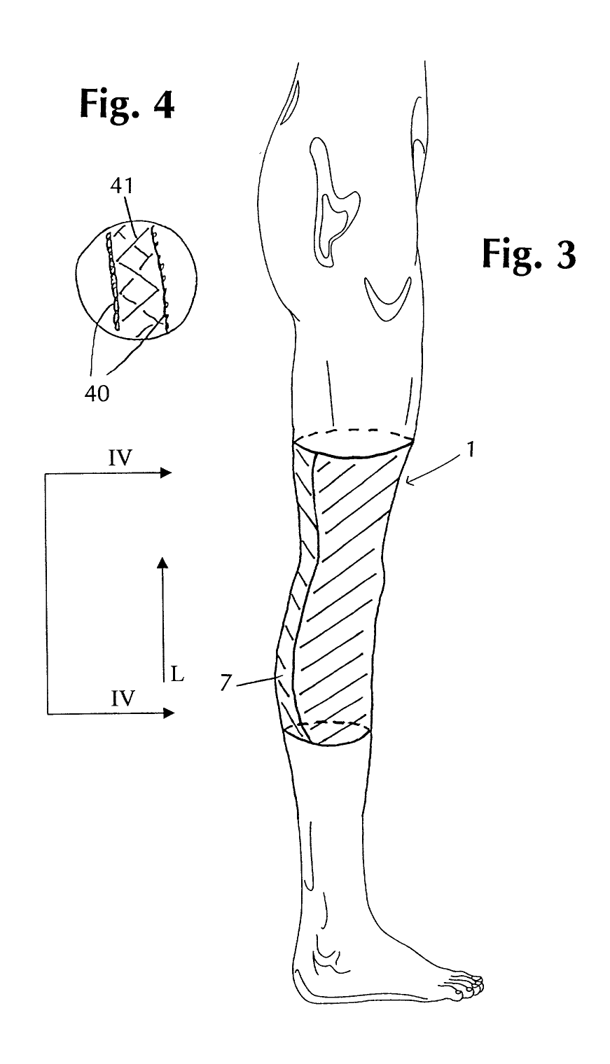

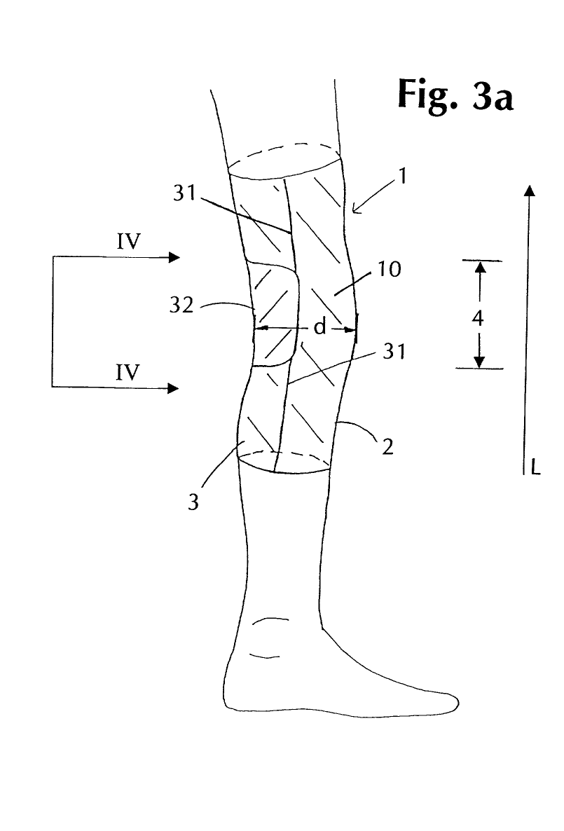

FIG. 2b illustrates a third embodiment of the bandage 1. The front side 2 and bending side 3 of the bandage 1 are substantially rectangular in the longitudinal direction L. The longitudinal sides 20 are curved towards each other or concave in the central section 4. The bending side 3 is divided into two essentially mirror-image like sections of possibly different sizes which together with the front side 2 form one-piece. As illustrated in FIG. 3a, the flat knitted fabric 10 is the one piece. The flat knitted fabric 10 has two oppositely arranged edges which are sewn together at flat seam 31 to form the bandage 1 which is elastic extending in the longitudinal direction L. Referring to FIGS. 2b and 3a jointly, the central section 4 has an opening 32 on the bending side 3. The opening 32 can be cutout or punched out of the bending side 3 in the area of the joint bend. The opening 32 has the general shape of a rectangle with edges 33. In a preferred embodiment, the opening 32 extends at least halfway around the leg of the patient or user. As seen in FIG. 2b, the edges 33 are curved away from each other in a direction transverse to the longitudinal direction L of the bandage 1. The size of the opening 32 in the longitudinal direction L approximates the diameter d of the bandage 1 and, when the joint or knee is moved, the change in the size of the opening in that direction approximates the change in size of dimension d. This results, in part, because the central section 4 is only slightly stretched by such movement.

In the FIG. 2b embodiment, the opening 32 is filled by the strip-like twin-layer knitted spacer fabric 7 for conducting moisture such as sweat. The strip-like twin-layer knitted spacer fabric 7 is sewn into the bandage 1 with a flat seam (not shown) surrounding the opening 32. Further, prior art bandages are prone to folds in the hollow of the knee 50. The folds typically extend transverse to the longitudinal direction of the bandage 1. In this embodiment, the opening 32 is constructed so that the area prone to folds in the bending position is covered by the moisture-conducting strip-like twin-layer knitted spacer fabric 7.

Where appropriate an elastic covering may be included in the hose openings, such as opening 32 shown in FIG. 2b.

Referring now to FIG. 4, the construction of the twin-layer knitted spacer fabric 7 will be described. The twin-layer knitted spacer fabric 7 is constructed to be bielastic. Two layers 40 contain polyamide 33 f12 and LYCRA® 44 dtex. LYCRA® is a registered trademark of E. I. duPont de Nemours and Company Corporation. The two layers 40 are advantageously spaced a distance of 2 to 3 mm. Outer surfaces of the two layers 40 are provided with small patterns. Spaced connecting threads 41 are located between the layers 40. The connecting threads 41 conveniently extend at an angle of approximately 45° with respect to the layers 40. The connecting threads 41 contain polyester and a moisture-conducting system. The twin-layer knitted spacer fabric 7 intended for application in the joint bend transports moisture from the layer directly in contact with the skin of the patient via the moisture-conducting connecting threads 41 to the layer of the strip-like twin-layer knitted spacer fabric 7 remote from the body running essentially parallel to the layer on the body side.

In a further advantageous arrangement, the strip-like twin-layer knitted spacer fabric 7 is constructed of Polyester, Polyamide and/or LYCRA. The material used to manufacture the strip-like twin-layer knitted spacer fabric 7 will determine the stretchability or elasticity of the bandage 1. Depending on the knitting structure chosen, the knitted spacer fabric may be of low stretchability or specifically elastic in accordance with the use to which it is intended. The strip-like twin-layer knitted spacer fabric 7 can be manufactured with different elasticity in the longitudinal direction and a direction transverse thereto. The knitted spacer fabric 7 manufactured from such materials are easily maintained, durable and comfortable.

While preferred embodiments have been illustrated and described, this has been by way of illustration and the invention should not be limited except as required by the scope of the appended claims.

What is claimed is:

1. An elastic hose-shaped bandage having a front side and a bending side, comprising:
a twin-layer knitted spacer fabric attached to the bending side of the elastic hose-shaped bandage for conducting moisture away from the skin.

2. An elastic hose-shaped bandage according to claim 1, wherein the twin-layer knitted spacer fabric extends over an entire longitudinal length of the elastic hose-shaped bandage.

3. An elastic hose-shaped bandage according to claim 2, further comprising:
a central region in the twin-layer knitted spacer fabric.

4. An elastic hose-shaped bandage according to claim 3, wherein the strip-like twin-layer knitted spacer fabric has an expanded width in the central section.

5. An elastic hose-shaped bandage according to claim 1, further comprising: a central region in the elastic hose-shaped bandage located at a bending side thereof and having an opening in the central region; and wherein the twin-layer knitted spacer fabric is located in the opening.

6. An elastic hose-shaped bandage according to claim 5, wherein the opening extends about half-way around the elastic hose-shaped bandage.

7. An elastic hose-shaped bandage according to claim 5, wherein the opening is generally rectangularly shaped and has edges curved away from each other and substantially transverse to a longitudinal direction of the elastic hose-shaped bandage.

8. An elastic hose-shaped bandage according to claim 5, wherein the elastic hose-shaped bandage has a diameter, and wherein the opening has a size which changes in the longitudinal direction an amount generally corresponding to a change in the diameter of the elastic hose-shaped bandage when a bandaged joint of a wearer is flexed.

9. An elastic hose-shaped bandage according to claim 1, wherein the elastic hose-shaped bandage is made of a flat knitted fabric.

10. An elastic hose-shaped bandage according to claim 9, wherein the flat knitted fabric is made of substantially one half of crimped Nylon yarn and substantially one half of rubber and elastic fibers.

11. An elastic hose-shaped bandage according to claim 9, wherein the flat knitted fabric is made of substantially one half of crimped Nylon yarn and substantially one half of rubber and elastic fibers.

12. An elastic hose-shaped bandage according to claim 5, having a front portion and a bending side portion, and wherein the portions are joined by a flat seam, and wherein the opening is in the bending side portion and divides the bending side portion into two substantially similar sections.

13. An elastic hose-shaped bandage according to claim 1, wherein the twin-layer knitted spacer fabric is attached to the elastic hose-shaped bandage by a flat seam.

14. An elastic hose-shaped bandage according to claim 1, wherein the twin-layer knitted spacer fabric has two layers spaced about 2 to 3 mm from each another; and a plurality of spaced connecting threads located between the two layers.

15. An elastic hose-shaped bandage according to claim 14, wherein the connecting threads are disposed at approximately 45° with respect to the two layers for conducting moisture.

16. An elastic hose-shaped bandage according to claim 1, wherein the strip-like twin-layer knitted spacer fabric is made of a material selected from polyester, polyamide, Lycra, and combinations thereof.
